(19) United States
(12) Patent Application Publication
(10) Pub. No.: US 2003/0133945 A1
(43) Pub. Date: Jul. 17, 2003

(54) Natural food supplement

(76) Inventor: Michael Donald Farley, Indialantic, FL (US)

Correspondence Address: Bidyut K. Niyogi, Transnational Enterprises, Inc., Suite #207, 95 Bulldog Blvd., Melbourne, FL, 32901, US

(21) Appl. No.: 10/045,824

(22) Filed: Jan. 11, 2002

(51) Int. Cl. A61K 35/78; A61K 31/7048; A61K 31/198; A61K 31/685; A61K 31/404; A61K 31/015; A61K 31/05

(52) U.S. Cl. 424/195.15; 424/729; 424/756; 514/27; 514/414; 514/561; 514/456; 514/733; 514/78; 514/765

(57) ABSTRACT

The present invention is a food supplement for a human body's natural immune function, formed from natural occurring substances in the form of a broth. The food supplement consists of the following:

(a) 1 to 35 mg. per mnilliliter—Apigenin

(b) 200 to 600 mg.—Chrysin

(c) 200 to 600 mg.—Colorius Versicolor

(d) 50 to 150 mg.—3,3′ Diindolymethane (DIM)

(e) 50 to 150 mg.—Resveratrol 25%

(f) 50 to 150 mg.—Turmeric Extract 95%

(g) 40 to 140 mg.—Green Tea Extract 95%

(h) 20 to 80 mg.—Quercitin Dihydrate 99%

(i) 15 to 75 mg.—Phosphtidyl Choline 50%

(j) Trace mineral selenium-L—10 to 60 mgs. L-Selenium Methionine (5000 mcg/gr)

It may be advantageous to also include 25 mg to about 150 mg. of Myricetin.

This invention relates a natural occurring food supplement for a human body's natural immune function, so as to be useful in the treatment of numerous medical problems, for the human being, specifically cancer.

### SUMMARY OF THE INVENTION

The present invention utilizes natural occurring food constituents that are available in abundance and specifically formulated as a broth type food supplement. This food supplement formula as a broth will assist a human body's natural immune function so as to fight serious medical and health problems. The formula consists in composition per unit dose in liquid or capsule gel caplets.

The composition of the formula per unit dose, preferably, in liquid form as a broth and includes:

(a) Apigenin—1 to 35 mgs per milliliter

(b) Chrysin—200 to 600 mg

(c) Clorius Versicolor—200 to 600 mg

(d) 3,3′ Diindolymethane (DIM)—50 to 150 mg

(e) Resveratrol 25%—50 to 150 mg

(f) Turmeric Extract 95%—40 to 140 mg

(g) Green Tea Extract 95%—40 to 140 mg

(h) Quercitin Dihydrate 99%—20 to 80 mg

(i) L-Selinium Methionine—Trace mineral selenium—10 to 60 mg

The natural occurring constituents are available from the following:

Food form—Broth

Apigenin is a flavonoid abundantly present in fruits and vegetables.

Chrysin is isolated from Chrysanthemum morifolium.

Colorius Versicolor is an edible mushroom and is the source of Polysacaride Krestin (PSK).

3,3′ Diindolymethane (DIM) is a component of cruciferous vegetables.

Resveratrol is a phytochemical found in grapes in high concentrations.

Tumeric is a well-known spice, and is another name for curcumin.

Green Tea Extract is taken from green tea leaves, and is a popular beverage.

Quercitin is abundantly present in many fruits and vegetables.

L-Selinium Methionine is a form of the trace mineral selenium found in brewer's yeast and wheat germ, liver, butter, fish and shellfish.

Advantageously, the food supplement may also include per dose unit 25 mg to 150 mg of Myricentin. Moreover, the food supplement also may include per dose unit 15 to 75 mg of Phosphtidyl Choline 50%.

An object of the present invention, is to provide a natural occurring food supplement, from abundantly available constituents, for example, Apigerin is a flavonoid present in abundance in fruits and vegetables.

A further object of the present invention is that in a liquid form as a broth type food supplement, it is easily introduced into the human body, so as to stave off, very serious medical situations such as cancer and other health problems associated with the body's immune function.

The present invention will now be described, only by way of example, and is in no way limited to this example.

The present invention is a food supplement formula for a human body's natural immune function, so that it can fight off, serious illness such as cancer, and other related medical conditions. The food supplement is in a broth form, and the composition of the formula per unit dose in liquid form is as follows:

(a) Apigenin—1 to 35 mgs. per milliliter

(b) Chrysin—200 to 600 mg.

(c) Clorius Versicolor—200 to 600 mg.

(d) 3,3′ Diindolymethane (DIM)—50 to 150 mg

(e) Resveratrol 25%—50 to 150 mg.

(f) Turmeric Extract 95%—50 to 150 mg.

(g) Green Tea Extract 95%—40 to 140 mg.

(h) Quercitin Dihydrate 99%—20 to 80 mg.

(i) L-Selinium Methionine—Trace mineral selenium—10 to 60 mg.

Advantageously, the food supplement broth may also include per dose unit of about 25 mg. to about 150 mg. of Myricertin. Moreover, the food supplement also may include per dose unit 25 mg. to 150 mg. of Phosphitidyl Choline 50%

In the present invention, the food supplement specifically consists in the amount per unit dose as follows:

(a) 10 mg. Apigenin

(b) 400 mg. Chrysin

(c) 400 mg. Colorius Versicolor 20% Polysacride Krestin (PSK)

(d) 100 mg. 3,3′ Diindolymethane (DIM)

(e) 100 mg. Resveratrol 25%

(f) 100 mg. Turmeric Extract 95%

(g) 89.5 mg. Green Tea Extract 95%

(h) 50 mg. Quercitin Dihydrate 99%

(i) 33.3 mcg. L-Selenium Methionine (5000 mcg/gr)

Suitably, the food supplement may also include in each dose per unit, of about 100 mg. of Myricentin.

Conveniently, the food supplement may also include in each dose per unit of 40 mg. Phosphtidyl Choline 50%.

Since the food supplement is easily introduced into the human body in liquid form and the constituents available in abundance in natural occurring substances, such as fruits and vegetables, turmeric and green tea extract, no side effects appear to occur to the human body, or to its immune function.

1. A food supplement formula for a human body's natural immune function, the composition of the formula per unit dose in liquid or capsule gel caplets consists of:
(a) Apigenin—1 to 35 mgs. per milliliter
(b) Chrysin—200 to 600 mg
(c) Clorius Versicolor—200 to 600 mg
(d) 3,3′ Diindolymethane (DIM)—50 to 150 mg
(e) Resveratrol 25%—50 to 150 mg
(f) Turmeric Extract 95%—50 to 150 mg
(g) Green Tea Extract 95%—40 to 140 mg
(h) Quercitin Dihydrate 99%—20 to 80
(i) L-Selinium Methionine—Trace mineral selenium—10 to 60 mg.

2. A formula as claimed in claim 1, characterized in that each per dose unit also includes 25 mg to about 150 mgs. of Myricertin.

3. A formula as claimed in claim 1, characterized in that each dose per unit also includes Phosphtidyl Choline 50% —15 to 75 mg.

4. A food supplement formula for a human body's natural immune function, as claimed in claim 1, characterized in that the amount in per unit dose form consists of:
(a) 10 mg. Apigenin
(b) 400 mg. chrysin
(c) 400 mg. Colorius Versicolor 20% Polysacride Krestin (PSK)
(d) 100 mg. 3,3′ Diindolymethane (DIM)
(e) 100 mg. Resveratrol 25%
(f) 100 mg. Turmeric Extract 95%
(g) 89.5 mg. Green Tea Extract 95%
(h) 50 mg. Quercitin Dihydrate 99%
(i) 33.3 mcg. L-Selenium Methionine (5000 mcg/gr)

5. A formula per unit dose as claimed in claim 4, characterized in that each per dose unit also includes 100 mg. Myricetin.

6. A formula as claimed in claim 4, characterized in that each dose per unit also includes 40 mg. Phosphtidyl Choline 50%.

7. A food supplement formula for a human body's natural immune function, the composition of the formula per unit dose in liquid or capsule gel caplets consists of:
(a) 1 to 35 mg. per milliliter—Apigenin
(b) 200 to 600 mg.—Chrysin
(c) 200 to 600 mg.—Colorius Versicolor
(d) 50 to 150 mg.—3,3′ Diindolymethane (DIM)
(e) 50 to 150 mg.—Resveratrol 25%
(f) 50 to 150 mg.—Turmeric Extract 95%
(g) 40 to 140 mg.—Green tea Extract 95%
(h) 20 to 80 mg.—Quercitin Dihydrate 99%
(i) 15 to 75 mg.—Phosphtidyl Choline 50%
(j) Trace mineral selenium—L—10 to 60 mgs L-Selenium Methionine (5000 mcg/gr)

8. A food supplement formula for a human body's natural immune function, the composition of the formula per unit dose in liquid or capsule gel caplets consists of:
(a) 10 mg.—Apigenin
(b) 400 mg.—Chrysin
(c) 400 mg.—Colorius Versicolor 20% Polysecride kristin (PSK)
(d) 100 mg.—3,3′ Diindolymethane (DIM)
(e) 100 mg.—Resveratrol 25%
(f) 100 mg.—Turmeric Extract 95%
(g) 89.5 mg.—Green Tea Extract 95%
(h) 50 mg.—Quercitin Dihydrate 99%
(i) 40 mg.—Phosphtidyl Choline 50%
(j) 33.3 mg.—L-Selinium Methionine (5000 mcg/gr)

9. A food supplement formula for a human body's natural immune function, the composition of the formula per unit dose in liquid or capsule gel caplets consists of:
(a) 1 to 35 mg. per milliliter—Apigenin
(b) 200 to 600 mg.—Chrysin
(c) 200 to 600 mg.—Colorius Versicolor
(d) 50 to 150 mg.—3.3′ Diindolymethane (DIM)
(e) 50 to 150 mg.—Resveratrol 25%
(f) 50 to 150 mg.—Turmeric Extract 95%
(g) 40 to 140 mg.—Green Tea Extract 95%
(h) 20 to 80 mg.—Quercitin Dihydrate 99%
(i) 15 to 75 mg.—Phosphtidyl Choline 50%
(j) Trace mineral selenium—L—10 to 60 mgs. L-Selenium Methionine (5000 mcg/gr)
(k) 25 to about 150 mg. Myricetin

10. A food supplement formula for a human body's natural immune function, the composition of the formula per unit dose in liquid or capsule gel caplets consists of:
(a) 10 mg.—Apignin
(b) 400 mg.—Chrysin
(c) 400 mg.—Colorius Versicolor 20% Polysecride Krestin (PSK)
(d) 100 mg.—3,3′ Diindolymethane (DIM)
(e) 100 mg.—Resveratrol 25%
(f) 100 mg.—Turmeric Extract 95%
(g) 89.5 mg.—Green Tea Extract 95%
(h) 50 mg.—Quercitin Dihydrate 99%
(i) 40 mg.—Phosphtidyl Choline 50%
(j) 33.3 mg.—L-Selinium Methionine (5000 mcg/gr)
(k) 100 mg.—Myricetin
